(19) United States
(12) Patent Application Publication
(10) Pub. No.: US 2021/0307656 A1
(43) Pub. Date: Oct. 7, 2021

(54) METHODS AND SYSTEMS FOR DETERMINING HEARING THRESHOLDS

(71) Applicant: THE BIONICS INSTITUTE OF AUSTRALIA, East Melbourne, Victoria (AU)

(72) Inventors: Darren Mao, Melbourne, Victoria (AU); Hamish Innes-Brown, Melbourne, Victoria (AU); Matthew A. Petoe, Melbourne, Victoria (AU); Yan T. Wong, Melbourne, Victoria (AU); Colette M. McKay, Melbourne, Victoria (AU)

(21) Appl. No.: 17/268,956

(22) PCT Filed: Aug. 16, 2019
(86) PCT No.: PCT/AU2019/050862

§ 371 Date: Feb. 16, 2021

(30) Foreign Application Priority Data
Aug. 17, 2018 (AU) 2018903020

(51) Int. Cl. A61B 5/12; A61B 5/38; A61B 5/00

(52) U.S. Cl. CPC A61B 5/125; A61B 5/7203; A61B 5/38

(57) ABSTRACT

Embodiments generally relate to a method of determining a hearing threshold value for a subject. The method comprises receiving at least one first response signal relating to an aural stimulation experienced by the subject at a first intensity level; receiving at least one second response signal relating to an aural stimulation experienced by the subject at a second intensity level, wherein the second intensity level is below an expected hearing threshold for the subject; calculating at least one first value relating to the at least one first response signal; calculating a second value relating to the at least one second response signal; plotting the at least one first value against the first intensity level on a plot; calculating a regression curve that fits the plot; and determine the hearing threshold of the subject to be the intensity level that corresponds to the second value along the regression curve.

### TECHNICAL FIELD

Embodiments generally relate to methods, devices and systems for hearing assessment. In particular, described embodiments are directed to methods, devices and systems for objective assessment of hearing thresholds.

### BACKGROUND

Accurate assessment of hearing is important for screening and diagnosis of hearing impairment and also for validation of hearing instrument fitting. In the case of a hearing instrument, it is important to know whether the instrument has been adjusted so that an appropriate range of sound levels (such as those typical of speech) are audible and not too loud when the hearing instrument is worn. For example, a cochlear implant may have 22 stimulating electrodes, each of which require calibration with electrical parameters to cause the cochlear implant to operate within a hearing threshold and a maximum comfortable loudness.

Hearing assessments to determine the range of sound stimulus levels that elicit sound percepts in patients between thresholds of hearing and uncomfortably loud sounds are normally determined using behavioural tasks. For example, a patient may be asked to listen to a sound recording, and press on a button or otherwise give an indication of when they can hear a tone. By playing tones of various intensities, the patient's hearing range can be determined. However, these tests are subjective, requiring a behavioural response from the patient. This can lead to inaccuracies in determining hearing ranges, particularly for people who may have difficult following instructions or performing the behavioural task, such as infants and cognitively impaired persons.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

It is desired to address or ameliorate one or more shortcomings or disadvantages associated with prior systems for hearing assessment, or to at least provide a useful alternative thereto.

### SUMMARY

Some embodiments relate to a method of determining a hearing threshold value for a subject, the method comprising:
receiving at least one first response signal relating to an aural stimulation experienced by the subject at a first intensity level;
receiving at least one second response signal relating to an aural stimulation experienced by the subject at a second intensity level, wherein the second intensity level is below an expected hearing threshold for the subject;
calculating at least one first value relating to the at least one first response signal;
calculating a second value relating to the at least one second response signal;
plotting the at least one first value against the first intensity level on a plot;
calculating a regression curve that fits the plot; and
determine the hearing threshold of the subject to be the intensity level that corresponds to the second value along the regression curve.

According to some embodiments, the at least one first value is a phase-locking value (PLV) of the at least one first response signal, and wherein the second value is a phase-locking value (PLV) of the at least one second response signal.

In some embodiments, the at least one first value is a peak-to-peak amplitude of the at least one first response signal, and wherein the second value is a peak-to-peak amplitude of the at least one second response signal.

According to some embodiments, the at least one first value is a root mean square (RMS) value of the at least one first response signal, and wherein the second value is a root mean square (RMS) value of the at least one second response signal.

In some embodiments, the at least one first value is a peak power value of the at least one first response signal, and wherein the second value is a peak spectral power value of the at least one second response signal.

According to some embodiments, the second intensity level corresponds to an absence of aural stimulation, and the second value is a baseline feature value.

According to some embodiments, the baseline feature value is extracted so that the noise level of the baseline feature value is within 0.05 arbitrary units of PLV of the noise level of the aural stimulation. According to some embodiments, the noise level of the baseline feature value is within 0.01 arbitrary units of PLV of the noise level of the aural stimulation.

Some embodiments further comprise establishing upper and lower bounds of the at least one first value, wherein the upper and lower bounds are proportionate to the noise level of the at least one first response. According to some embodiments, the upper and lower bounds of the at least one first value are determined to be one standard deviation away from the median of the bootstrap distribution of the at least one first value. In some embodiments, the upper and lower bounds of the at least one first value are determined to be first and third quartiles of the bootstrap distribution of the at least one first value.

Some embodiments further comprise modifying the upper and lower bounds based on their proximity to the baseline feature value. Some embodiments further comprise replacing any lower bounds that are below the baseline feature value with negative infinity.

In some embodiments, the step of calculating a regression curve that fits the plot comprises calculating at least one growth function within a defined parameter space. Some embodiments further comprise excluding any growth functions that exceed the lower and upper bounds. Some embodiments further comprise applying a weight to each growth function based on the upper and lower bounds.

According to some embodiments, the at least one first response signal is received from an electrode located on the body of the subject. In some embodiments, the electrode is located on the head of the subject.

According to some embodiments, the at least one first response signal is received from an electrode located on a hearing device worn by the subject.

In some embodiments, the at least one first response signal and the at least one second response signal relate to electrical activity of the subject's brain. In some embodiments, the at least one first response signal and the at least one second response signal are electroencephalography (EEG) signals.

Some embodiments further comprise delivering the aural stimulation. In some embodiments, the aural stimulation is delivered for a period of between 5 ms and 100 ms. In some embodiments, the aural stimulation is delivered at a first intensity level of between −10 dB SPL and 120 dB SPL.

According to some embodiments, the second intensity level is delivered for a period of between 0.1 seconds and 10 seconds.

In some embodiments, receiving at least one first response signal relating to an aural stimulation experienced by the subject at a first intensity level comprises receiving a plurality of first response signals relating to aural stimulation experienced by the subject at a plurality of different intensity levels.

Some embodiments further comprise repeating the steps of the method with additional epochs of response signals until a stopping criterion is met. According to some embodiments, the additional epochs are added to ensure that the same number of epochs exist at every stimulation intensity level. In some embodiments, the additional epochs are added to ensure that the number of epochs at every stimulation intensity level is within a predefined limit of the number of epochs that exist for every other stimulation intensity level. According to some embodiments, the predefined limit is 10.

In some embodiments, the additional epochs are added to ensure that the noise level is within 0.05 arbitrary units of PLV at every stimulation intensity. In some embodiments, the additional epochs are added to ensure that the noise level is within 0.01 arbitrary units of PLV at every stimulation intensity

According to some embodiments, hearing thresholds are determined for more than one aural stimulus at a time, by performing the method steps for each aural stimulus interleaved with the method steps for at least one other aural stimulus.

Some embodiments relate to a system for determining a hearing threshold value for a subject, the system comprising:
a hearing assessment device configured to perform the method of some other embodiments.

Some embodiments further comprise a stimulation member configured to provide the aural stimulation.

Some embodiments further comprise at least two electrodes configured to measure the at least one first response signal and the at least second first response signal and communicate the at least one first response signal and the at least second first response signal to the hearing assessment device. According to some embodiments, the electrodes are configured to be placed on a scalp of the subject.

According to some embodiments, the at least 2 electrodes comprise a reference electrode and a measuring electrode. In some embodiments, the at least 2 electrodes further comprise a ground electrode.

### BRIEF DESCRIPTION OF DRAWINGS

Embodiments are described in further detail below, by way of example and with reference to the accompanying drawings, in which:

FIG. 1 shows a block diagram of a hearing assessment system according to some embodiments;

FIG. 2 shows a flow diagram illustrating an example method using the system of FIG. 1;

FIG. 3 shows a flow diagram illustrating a method of upper bound estimation using the system of FIG. 1;

FIG. 4 shows a flow diagram illustrating a method of lower significant level threshold estimation using the system of FIG. 1;

FIG. 5 shows a flow diagram illustrating a method of growth function threshold estimation using the system of FIG. 1;

FIG. 6 shows an example graph of responses recorded by the system of FIG. 1 represented as a phase-locking value spectrogram;

FIGS. 7A and 7B show example graphs generated using the methods shown in FIGS. 3 to 5;

FIG. 8 shows an example graph illustrating the improvement in threshold estimation accuracy using the system of FIG. 1 with an increase in test time;

FIG. 9A shows an example graph of responses recorded by the system of FIG. 1 showing amplitude and RMS values;

FIG. 9B shows an example graph of responses recorded by the system of FIG. 1 represented as a peak spectral power value spectrogram;

FIG. 10A shows an example graph of growth functions showing upper and lower feature bounds and valid growth functions as determined by the system of FIG. 1;

FIG. 10B shows threshold estimates as determined by the system of FIG. 1;

FIG. 11 shows a graph illustrating how responses to untested stimulus intensities are predicted by the system of FIG. 1;

FIG. 12 shows an example graph illustrating the change in the expected phase-locking feature baseline and uncertainty over a number of epochs;

FIG. 13A shows an example graph of predicted bounds when adding a new stimulus intensity;

FIG. 13B shows an example graph of predicted bounds when adding epochs to existing stimulation intensities; and

FIG. 14 shows an example graph illustrating the improvement in threshold estimation accuracy using the system of FIG. 1 with an increase in test time.

### DETAILED DESCRIPTION

Embodiments generally relate to methods, devices and systems for hearing assessment. In particular, described embodiments are directed to methods, devices and systems for objective assessment of hearing thresholds.

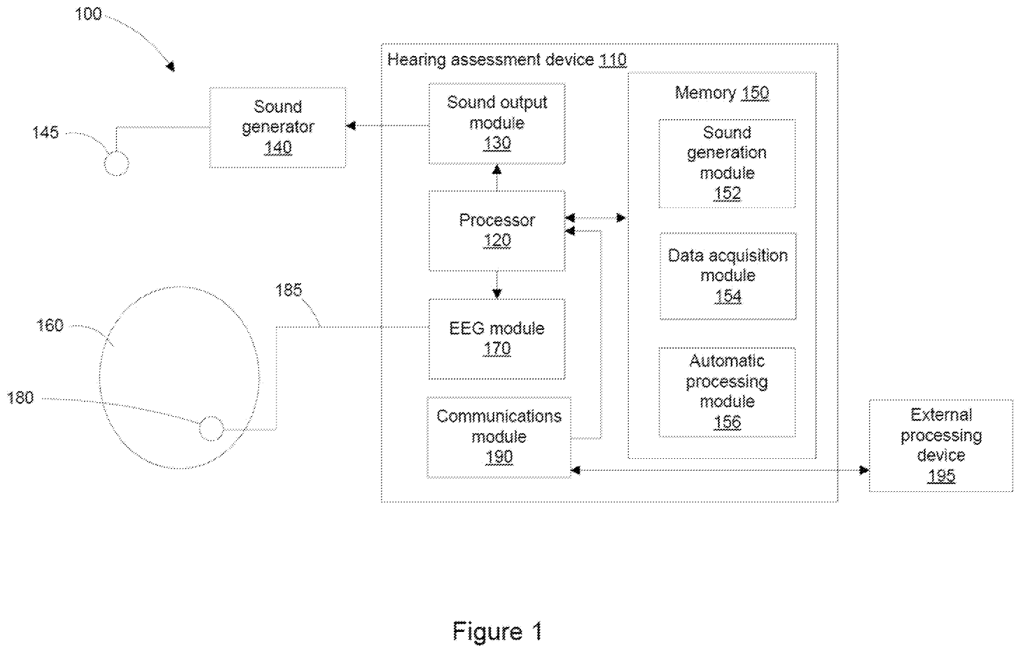

FIG. 1 shows a system 100 for hearing assessment using electroencephalography (EEG). EEG is an electrophysiological monitoring method to record electrical activity of the brain via non-invasive electrodes placed along a patient's scalp. EEG is used to measure voltage fluctuations in the brain. System 100 may be used to provide an objective method of assessing hearing thresholds in a patient.

System 100 is made up of a hearing assessment device 110, a sound generator 140, a stimulation member 145, and an external processing device 195. According to some embodiments, system 100 also comprises at least two electrodes 180 configured to be positioned on a scalp 160 of a patient.

Hearing assessment device 110 has a processor 120, which communicates with a sound output module 130, memory 150, an EEG module 170, and a communications module 190. In the illustrated embodiment, sound generator 140 is a separate unit from assessment device 110. However, in some embodiments, sound generator 140 may form part of hearing assessment device 110.

Stimulation member 145 may be a speaker, earphone, hearing aid, hearing instrument, implantable auditory prosthesis comprising implantable electrodes, cochlear implant, brain stem implant, auditory midbrain implant, or other component used to provide aural stimulation to a patient. Aural stimulation may include acoustic stimulation, which may be delivered by a speaker or headphone in some embodiments. Aural stimulation may also include electric stimulation of a patient's auditory system, which may be delivered by a cochlear implant, for example.

According to some embodiments, stimulation member 145 may be an audiometric insert earphone, such as the ER2 insert earphones by Etymotic Research. In some embodiments, stimulation member 145 may interface with another component, such as a hearing aid or cochlear implant sound processor, in order to provide aural stimulation to the patient. Sound generator 140 causes the stimulation member 145 to produce a range of aural stimulation signals to assess the patient's hearing. According to some embodiments, sound generator 140 may be a soundcard, or a speech processor. In some embodiments, the stimulation member 145 may be a cochlear implant, a hearing aid, an acoustic hearing implant or an implant electrode for electric hearing. In these cases, sound generator 140 generates and transmits the appropriate electric waveform signal or instructions for the patterns of electrical pulses to stimulation member 145. In some embodiments, stimulation member 145 may be a transducer.

Electrodes 180 may be a number of electrodes configured to take EEG readings. According to some embodiments, electrodes 180 may include pin-type or flat-type active electrodes, such as the pin-type or flat-type electrodes produced by BioSemi. According to some embodiments, electrodes 180 may include sintered silver-chloride (Ag—Cl) electrodes. Electrodes 180 may be active electrodes in some embodiments.

According to some embodiments, electrodes 180 may comprise at least two electrodes. At least one electrode 180 may be configured to be a measuring electrode, which may be configured to be placed on the vertex of the head of a patient. At least one electrode 180 may be configured to act as a reference electrode. The at least one reference electrode may be configured to be placed on the mastoid contralateral to the sound stimulation side of the patient. According to some embodiments, electrodes 180 may comprise at least three electrodes, with at least one further electrode 180 configured to operate as a ground electrode or a driven right leg electrode, to improve common-mode rejection.

Although only one external processing device 195 is shown, assessment device 110 may be in communication with more than one external processing device 195, which may in some embodiments be desktop or laptop computers, mobile or handheld computing devices, servers, distributed server networks, or other processing devices. According to some embodiments, external processing device 195 may be configured to perform some or all of the functions described below as being performed by processor 120.

Processor 120 may include one or more data processors for executing instructions, and may include one or more of a microprocessor, microcontroller-based platform, a suitable integrated circuit, and one or more application-specific integrated circuits (ASIC's).

Sound output module 130 is arranged to receive instructions from processor 120 and send signals to sound generator 140, causing sound generator 140 to provide signals to stimulation member 145. Where stimulation member 145 comprises a speaker or earphone, the signals may include an acoustic signal delivered via the earphone or speaker in the sound field. Where stimulation member 145 comprises a hearing instrument, the signals may comprise a digital sound file delivered via direct audio input to the hearing instrument. Where stimulation member 145 comprises an implantable auditory prosthesis, the signals may comprise instructions for an electrical signal to be delivered by implanted electrodes in the implantable auditory prostheses.

Memory 150 may include one or more memory storage locations, either internal or external to system 100, and may be in the form of ROM, RAM, flash or other memory types. Memory 150 is arranged to be accessible to processor 120, and contain program code that is executable by processor 120, in the form of executable code modules. These may include sound generation module 152, data acquisition module 154, and automatic processing module 156.

EEG module 170 is configured to receive instructions from processor 120 and send signals to electrodes 180 via transmission channel 185, causing electrodes 180 to obtain EEG readings through scalp 160 of the patient. Communications module 190 may allow for wired or wireless communication between assessment device 110 and external processing device 195, and may utilise Wi-Fi, USB, Bluetooth, or other communications protocols.

System 100 may be used to determine the range of sound stimulus levels that elicit sound percepts in patients between their threshold of hearing and uncomfortably loud sounds. In particular, system 100 may be used to automatically determine a patient's threshold hearing levels. Processor 120 may be configured to execute instructions read from sound generation module 152 of memory 150, to cause processor 120 to send instructions to sound output module 130. Sound output module 130 may consequently communicate with sound generator 140, to cause sound generator 140 to generate a sound signal based on the instructions received. Sound generator 140 may output the sound signal to stimulation member 145 to cause stimulation member 145 to produce one or more sounds.

According to some embodiments, sound generator 140 may be configured to generate alternating periods of sounds and silence. Periods of sound may be 5 to 100 milliseconds in duration, and the periods of silence may be between 0.1 and 10 seconds in duration according to some embodiments. According to some embodiments, periods of sound may be 20 to 100 milliseconds in duration, and the periods of silence may be between 1.35 and 1.65 seconds in duration according to some embodiments. According to some embodiments, the periods of silence may be substituted with periods of sound played at a low intensity, determined to be lower than the expected hearing threshold of the patient.

Sound generator 140 may be configured to generate sounds with varying levels of intensity or loudness. For example, for a person with normal hearing, the sound level may be adjustable between approximately −10 and 120 dB sound pressure level (SPL). For a cochlear implant recipient, the sound level may be adjustable between their devices' limitations, namely 0 to 255 current levels (CLs) for Nucleus devices, for example. The CLs are related to units of current, or micro amps, according to formulas specific to devices and manufacturers. In the present document, stimulation levels will be measured in units of decibels relative to 1 μA, or dB-re-1 μA. Using these units, a stimulation of 1 micro-amp is 0 dB, and any other stimuli are scaled accordingly. The characteristics of the sound (for example, bandwidth, frequency, or amplitude) may be adjustable depending on the person being tested and the purpose of the testing.

Stimulation member 145 may be positioned on or near a patient, in order to aurally stimulate the patient. Electrodes 180 may be positioned in proximity to the vertex of the patient's head. Alternatively, electrodes 180 may be placed elsewhere on the patient's head.

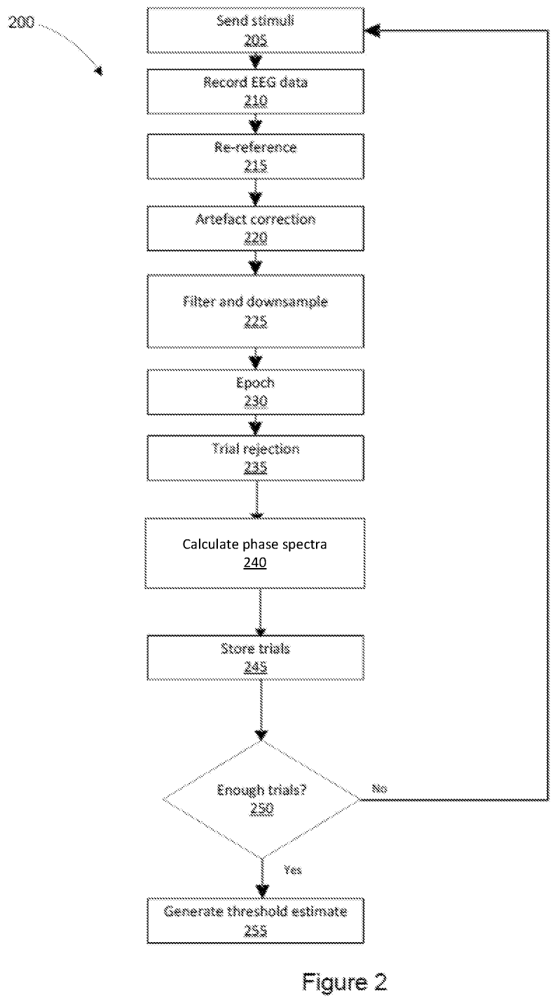

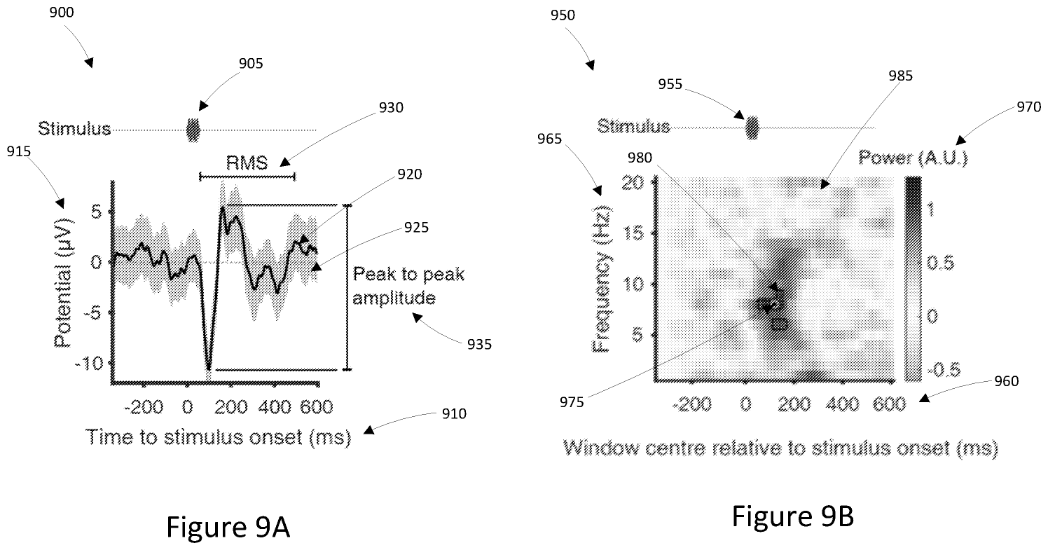

According to some embodiments, electrodes 180 may be placed in close proximity to a patient's head, such as on a hearing device, which may be a behind-the-ear worn hearing aid or a cochlear implant sound processor, for example. According to some embodiments, electrodes 180 may be located on an implanted device, such as by being incorporated into an implanted stimulator, for example. Where electrodes 180 are positioned on the external module of a hearing device or implanted within a cochlear implant, the orientation of electrodes 180 may be suboptimal for recording traditional N1-P2 response amplitude, as the direction of electrodes 180 may not align well with the expected field potential generated within the primary auditory cortex. Additionally, the distance between active and reference electrodes 180 will be small if they are both located on or near a hearing device. Both these issues lead to smaller amplitude responses recorded by electrodes 180, and responses that may not be accurately quantified by amplitude-based features when using traditional analysis. However, using the method described below with reference to FIGS. 2 to 5, electrodes 180 can be placed on an internal or external component of a hearing device. The method described with reference to FIGS. 2 to 5 uses phase locking value (PLV) features, so that the responses are affected less by recording location than when using traditional techniques that see a reduction in response amplitude when electrodes are not aligned optimally to the electric field generated in the brain in response to a stimulus. According to some embodiments, and as described below with reference to FIGS. 9A and 9B, the method may also or alternatively use other features of the response, such as the peak-to-peak amplitude, root mean square (RMS), or spectral power of the response.

When the patient hears a sound due to the stimulation provided by stimulation member 145, the neural activity in the patient's brain at or around the auditory cortex increases, leading to an increase in electrical voltage recorded by electrodes 180.

Data collected by detector electrodes 180 is carried by transmission channel 185 to EEG module 170, which communicates with processor 120. In some cases, the data may be stored in memory 150 for future processing by processor 120 and/or external computing device 195. In some embodiments, the data may be processed by processor 120 and/or external processing device 195 in real time.

Processor 120 may execute data acquisition module 154 to collect response data recorded by electrodes 180 in response to stimulation delivered to stimulation member 145. Methods 200 and 300, described in further detail below, may be performed by processor 120 executing data acquisition module 154.

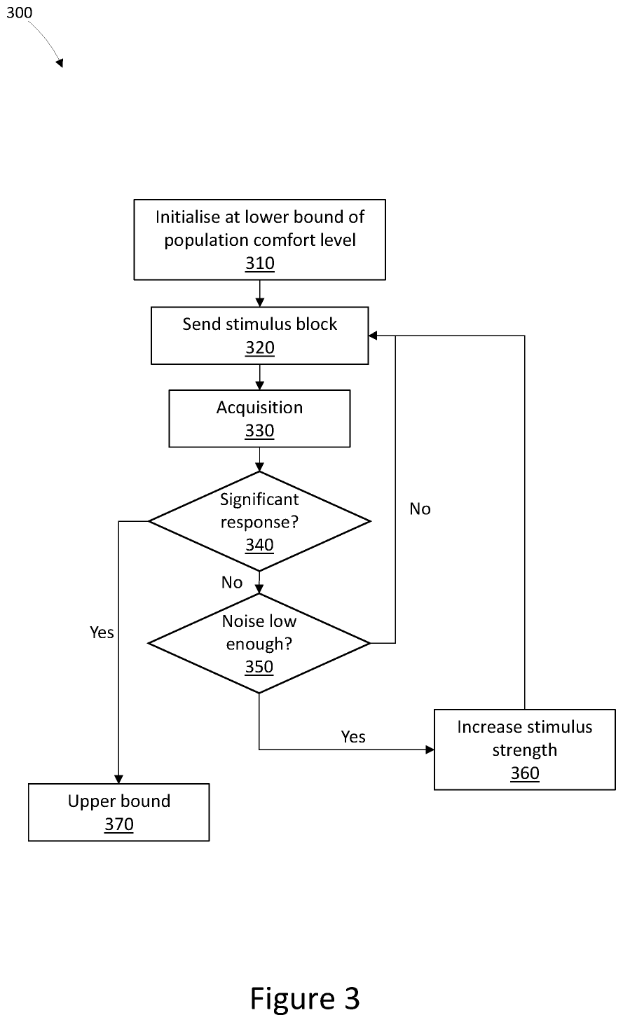

As described in further detail below with reference to FIGS. 3 to 5, processor 120 may subsequently execute automatic processing module 156, which may automatically process the data collected by processor 120 executing data acquisition module 154, and determine a hearing threshold level for the patient based on the data acquired.

Sounds generated by sound generator 140 may include sounds within the human hearing range. For acoustic sounds, these may include pure or warble tones in the range of 125 Hz to 16 kHz, for example. Acoustic sounds may be generated between 20 and 100 ms long in duration in some embodiments. According to some embodiments, the sounds may be between 40 and 60 ms long. According to some embodiments, the sounds may be around 50 ms long. The sound duration may be selected to avoid artefacts from the stimulus signal overlapping with the cortical response from the patient. For sounds presented via electric stimulation, these may be trains of bi-phasic pulses with standard parameters, for example 900 pulses per second, with 25 μS pulse width and 8 μS inter-phase gap. Pulse trains between 20 and 100 ms long in duration may be generated in some embodiments. According to some embodiments, the pulse trains may be between 40 and 60 ms long. According to some embodiments, the pulse trains may be around 50 ms long. The pulse train duration may be selected to avoid artefacts from the stimulus signal overlapping with the cortical response from the patient. According to some embodiments, the pulse train that may vary in one or more fixed parameters including pulse duration, interphase gap, current amplitude and rate.

In operation, processor 120 executes sound generation module 152 to generate sound parameters that are passed to sound output module 130. Sound output module 130 causes sounds based on the parameters to be generated by sound generator 140, and delivered by stimulation member 145. Sounds are generated at a variety of intensity levels. In some embodiments, sound intensities may be automatically generated during the testing procedure depending on the response recorded by electrodes 180. According to some embodiments, sounds may be delivered at a series of pre-determined intensities. According to some embodiments, the initial sound intensity for a test session may be pre-determined based on a set of initial parameters generated based on information about the patient, and subsequent levels of stimulation may be automatically determined based on responses recorded by electrodes 180.

According to some embodiments, the intensity levels may vary between −10 dB SPL and 120 dB SPL. In some embodiments, the intensity levels may vary between 0 dB SPL and 120 dB SPL. In some embodiments, the intensity levels may vary between 5 dB SPL and 120 dB SPL. In some embodiments, the intensity levels may vary between 20 dB SPL and 80 dB SPL. According to some embodiments, sound may be delivered at a series of levels that are estimated to be around and above the patient's hearing threshold, such as 5 dB SPL, 10 dB SPL, 20 dB SPL, 40 dB SPL and 60 dB SPL, for example. When the patient is a cochlear implant recipient, intensity levels may be varied between 0 and 255 current levels for Nucleus devices. For non-Nucleus devices, intensity levels may be varied an amount that corresponds to being between 0 and 255 current levels for a Nucleus device.

In some modes of operation, as detailed by FIG. 2, EEG responses to the stimulation are captured by electrodes 180 and communicated to processor 120 executing data acquisition module 152. Processor 120 then executes automatic processing module 156 to calculate a phase-locking value (PLV) for each set of responses to one stimulus intensity level, and fits a regression curve to the data. Processor 120 further extrapolates the regression curve to determine a hearing threshold, by determining the stimulation intensity level that would represent a PLV in the patient equivalent to the PLV calculated when no stimulation is being delivered. This process is described in further detail below with reference to FIGS. 2 to 5. While FIGS. 2 to 5 relate to using PLVs, according to some embodiments, as described below with reference to FIGS. 9A and 9B, values such as the peak-to-peak amplitude, RMS, or spectral power of the responses may be used instead of the PLVs.

FIG. 2 is a flowchart illustrating a method 200 for calculating a threshold hearing estimate using a hearing assessment device 110. Method 200 may be executed by processor 120 executing pre-processing module 154 and automatic processing module 156.

At step 205, processor 120 instructs sound output module 130 to cause sound generator 140 to deliver a predetermined sound signal to stimulation member 145. The parameters of sound signal may be determined by processor 120 executing sound generation module 152 on the fly, or may be read by processor 120 from memory 150.

At step 210, processor 120 executes data acquisition module 154. Processor 152 receives EEG data from module 170 as sensed by electrodes 180 and transmitted via transmission channel 185. Electrodes 180 may be positioned on a scalp 160 of a patient, in a position that allows electrodes 180 to record the cortical auditory evoked potential in response to the stimuli delivered by stimulation member 145. According to some embodiments, electrodes 180 may be BioSemi Active II system electrodes. According to some embodiments, electrodes 180 may form part of a DC coupled data recording system. A DC coupled data recording system may allow for data acquisition without distorting stimulation artefacts, making the artefacts easier to remove. According to some embodiments, processor 120 may execute MATLAB code to perform data processing functions.

At step 220, processor 120 performs artefact cancellation or correction. This may involve substituting any segments of the data recording having stimulus artefacts with segments of the pre-stimulus data recording that are therefore free of stimulus artefacts. This process is designed to remove the artefact contaminated regions of the data recording. Depending on the hardware configuration of system 100, different durations of the data recording may need to be substituted. For example, according to some embodiments, a segment that is 100 ms before the stimulus onset until 50 ms after stimulus onset may be substituted.

At step 225, processor 120 performs filtering and down-sampling of the data. Filtering may include using a bandpass Butterworth filter. According to some embodiments, the cut-offs for the bandpass filter may be around 1 Hz and 40 Hz. According to some embodiments, the cut-offs for the bandpass filter may be around 1 Hz and 20 Hz. The selection of the cut-offs may be made based on expected response morphology, and cut-offs may be selected to reduce or eliminate line noise which typically occurs at around 50 Hz or 60 Hz.

The filter type may be selected based on the desired calculation speed, and the expected magnitude and phase response of the received data. A 4^{th} order may be used in some embodiments. In some embodiments, the filter may be adjusted, or an alternative filter may be used, to minimise processing time. A Butterworth type filter may be used when it is desirable to produce a flat phase response, as Butterworth filters tend to produce relatively flat phase responses compared to other infinite impulse response (IIR) filters. A flatter phase response may result in less distortion of the true response.

Down-sampling may also be performed on the data. According to some embodiments, down-sampling may be performed between 200 and 300 Hz, or to around 256 Hz. The down-sampling frequency may be selected to be a high enough resolution to view the temporal waveform, but a low enough resolution to allow for storage of the data in memory 150 when many trials are acquired within a short timeframe.

At step 230, epoching is performed. Epoching involves isolating responses to the individual stimuli from the continuous EEG recording. This may be done by taking a time segment from a duration before the stimulus onset and to a duration after the stimulus onset. For example, a time segment from 200 ms pre stimulus-onset to 600 ms post stimulus onset may be selected. According to some embodiments, the durations may be selected so that the segment, or epoch, contains the whole response to the stimuli. Epoching may done to reduce the size of the data to be stored in memory 150, and to make further processing of the data by processor 120 more convenient and efficient.

At step 235, certain response data is rejected. In particular, some epochs of response data may contain motion artefacts. These artefacts may be recognised based on whether the measured voltage exceeds a particular threshold value. For example, epochs containing voltages exceeding ±80 μV may be eliminated in some embodiments. In some embodiments, epochs containing voltages exceeding ±100 μV may be eliminated. In some embodiments, epochs containing voltages exceeding ±120 μV may be eliminated. The threshold values may be selected based on the inherent noise of the system, which may be measurable based on the EEG voltage variation measured by electrodes 180 when no stimuli are being delivered.

At step 240, phase spectra is calculated to transform the time-domain response into the time-frequency representation, and allowing phase locking values (PLVs) to be calculated based on the phase spectra. According to some embodiments, processor 120 may calculate short-time Fourier transforms for each epoch of response data. According to some embodiments, the short-time Fourier transforms may be calculated with a Hamming window. According to some embodiments, the Fourier transforms may be calculated with a Hamming window of between 200 ms and 600 ms. According to some embodiments, the Fourier transforms may be calculated with a Hamming window of around 400 ms. The Hamming window may use a step size of between 10 ms and 30 ms, and the step size may be around 20 ms in some embodiments.

At step 245, the trial data is stored in memory 150 by processor 120. According to some embodiments, the data may additionally or alternatively be stored on external processing device 195. The data may be stored in a matrix or database. According to some embodiments, data may be stored in RAM using a MATLAB script. According to some embodiments, the cosine and sine of the phase values may also be stored, alongside the processed epochs of response data, to speed up calculation of the phase locking values. The data may be stored in the time-domain. Where processing speed is of concern, and particularly where MATLAB is being used, it is preferred not to use cell arrays for storing data, as using cell arrays may significantly increase processing time in some embodiments.

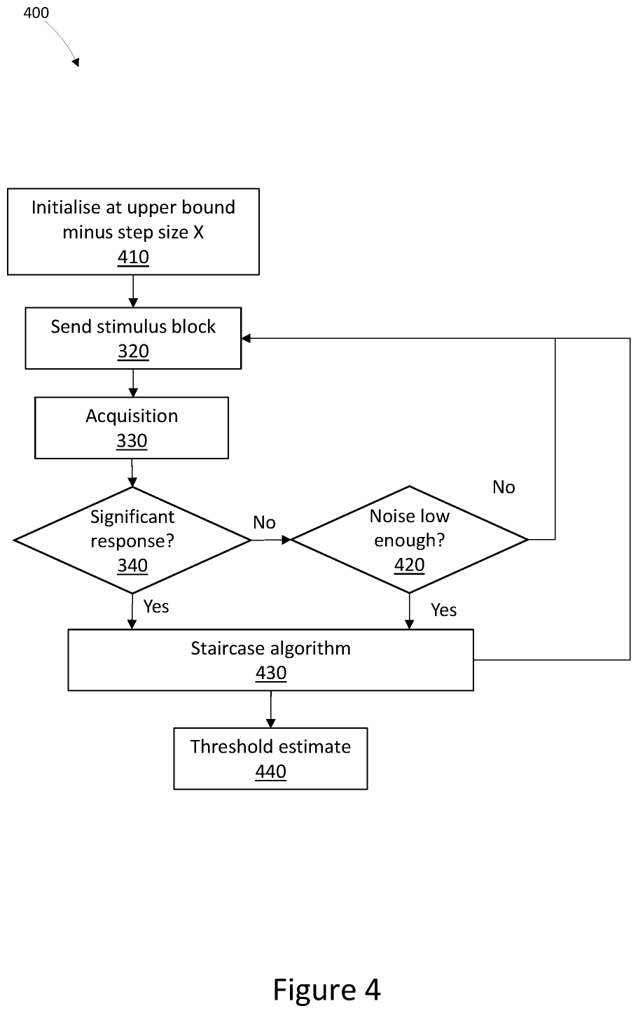

At step 250, processor 120 determines whether sufficient trials have been performed to estimate a hearing threshold, or whether more trials are required. If more trials are required, processor 120 may cause method 200 to start again from step 205. If no further trials are required, processor 225 generates the hearing threshold estimate based on the stored trial data. The process of determining the threshold based on the trial data is described in more detail below with reference to FIG. 4.

FIG. 3 is a flowchart illustrating a method 300 for upper bound estimation using system 100. Method 300 may be executed by processor 120 and/or external processing device 195, and is used to determine a set of stimulus parameters which is presumed audible by the patient or test subject. The upper bound parameter determined by method 300 may be used by both methods 400 and 500, as described in further detail below. Method 300 may be performed by processor 120 executing automatic processing module 156.

Method 300 begins at step 310, by processor 120 initialising stimulation parameters for an initial stimulation to be delivered by stimulation member 145 at a predetermined level. In some embodiments pertaining to acoustic stimulation, these parameters may be a constant 50 ms duration pure tone stimulation, with 4 ms linear up and down ramps. The stimulation level may be 40 dB HL for the specified frequency in some embodiments, where dB HL (decibels relative to average normal hearing level) is the average level of hearing for a young, healthy individual. In some alternative embodiments pertaining to electrical stimulation, the stimulation parameters may be a constant 50 ms duration, 900 pulse-per-second pulse train, with monopolar transmission of biphasic pulses at 25 microseconds pulse width and 8 microseconds interphase gap. The stimulus intensity, in units of dB-re-1 μA, may be the lower bound of the loud-but-comfortable level in a population of cochlear implant users.

At step 320, one stimulus block, which may be a block of stimulation pulse trains, is generated by sound output module 130 based on the current stimulation parameters, and delivered to the patient by stimulation member 145 via sound generator 140. In some embodiments pertaining to acoustic stimulation, the block may contain one presentation, or five to ten presentations of stimuli. In some embodiments pertaining to electrical stimulation, the block may contain one stimulation pulse train, or five to ten stimulation pulse trains. Where step 320 is performed directly after step 310, the stimulation parameters may be the predetermined parameters. Where step 320 is performed directly after step 360, the stimulation parameters may be the updated stimulus parameters as described below with reference to step 360.

At step 330, one or more response signals are received from electrodes 180 by EEG module 170, as described in further detail above with reference to step 210 of FIG. 2. The responses may be stored in the time-frequency domain in memory 150 by processor 120, in data blocks. According to some embodiments, the responses may be stored as frequency domain data, being the spectral phase data between 1 and 20 Hz and −400 ms to 600 ms post-stimulus. All responses acquired from the same stimulation parameters may appended into the same block in memory 150.

At step 340, the responses for one block of stimulation trials are tested for statistical significance by processor 120. In some embodiments, processor 120 may execute a Rayleigh test to determine the phase significance of the responses. A statistically significant response may be defined by a p value threshold. According to some embodiments, the threshold value may be a value of less than 0.05 or 0.001, so that only responses with a p value meeting below the threshold will be considered statistically significant by processor 120. A statistically significant response may signify an audible stimulus. Where a p value for a block of responses correlating to a particular stimulation intensity is less than the predetermined threshold, at step 370 the stimulation intensity is determined by processor 120 to be the upper bound value.

If the response is not determined by processor 120 to be significant at step 340, then at step 350, the responses' noise level is calculated by processor 120 to determine if it is low enough to indicate adequate data quality, and to conclude that there is no response at the current stimulation level. A noise level is deemed adequate in quality when the noise level drops below a predetermined threshold. In some embodiments, the threshold noise level may be determined by processor 120 to be when the PLV standard error is below 0.05 arbitrary units (A.U.) of PLV. As PLV is unitless, arbitrary units are used throughout the document to refer to PLV values. The PLV standard error may be calculated by processor 120 using a bootstrapping method. If the noise level is determined to not be low enough at step 350, more data is acquired at step 320.

If the noise level is determined to be low enough at step 350, processor 120 determines that no response to the stimulation intensity is being exhibited by the patient, and so the stimulus strength of the stimulation intensity level is increased at step 360. In some embodiments pertaining to acoustic stimulation, the increase may be achieved by changing the sound pressure level. For example, the sound may be increased by 20 dB. In some embodiments pertaining to electrical stimulation, the increase may be achieved via alteration of any of the parameters determined at step 310, which may include the pulse rate, stimulation mode, pulse duration, inter-phase gap and/or current level. For example, the current level may be stepped up by a constant 3 dB, while keeping other parameters constant, to achieve an increase in the stimulus strength. The method is then repeated from step 320 until an upper bound value is determined at step 370.

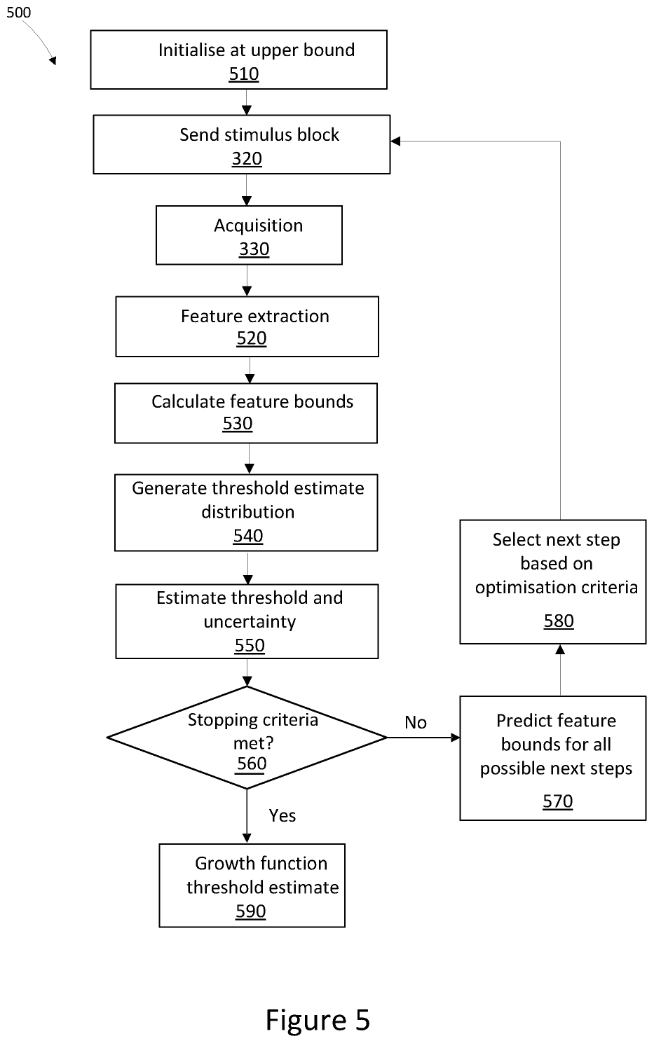

The upper bound level determined at step 370 is indicative of when a block of responses to a particular stimulation intensity level is statistically significant as calculated at step 340, and is the value of the stimulation intensity level when this occurs. The upper bound level determined at step 370 may be used as a guideline in methods 400 and 500, as described with reference to FIGS. 4 and 5, below. In some alternative embodiments, an upper bound level may be determined behaviorally or subjectively for use as a guideline in methods 400 and 500.

FIG. 4 is a flowchart illustrating a method 400. Method 400 is an adaptive procedure for estimating lowest significant level thresholds using system 100. Method 400 may be executed by processor 120 and/or external processing device 195. In particular, method 400 may be performed by processor 120 executing automatic processing module 156.

Method 400 begins at step 410, at which processor 120 initiates a starting stimulus level based on an upper bound value, which may be determined in method 300, as described above, or may be an upper bound that has been subjectively determined, for example from experience or from behavioural testing of stimulation intensity upper levels. In particular, processor 120 may set the starting stimulus level as the upper bound value minus an initial step size “X”. In some embodiments pertaining to acoustic stimulation, the initial step size X may be between 5 and 40 dB. In some embodiments, the initial step size may be around 20 dB. In some embodiments pertaining to electrical stimulation, the initial step size X may be between 1 and 5 dB. In some embodiments, the initial step size may be around 3 dB.

After initialising the stimulation level, processor 120 causes a stimulus block with the determined parameters to be generated and delivered to a patient at step 320. An EEG response to the stimulation is recorded by electrodes 180, and received by EEG module 170 at step 330. At step 340, processor determines whether or not the response received is a significant response. Steps 320, 330 and 340 are described in further detail above, with reference to FIG. 3.

If processor 120 determines that there is not a significant response at step 340, then processor 120 proceeds to execute step 420, at which the responses' noise level is calculated by processor 120 to determine if it is low enough to indicate adequate data quality, and to conclude that there is no response at the current stimulation level. A noise level is deemed adequate in quality when the noise level drops below a predetermined threshold. In some embodiments, the threshold noise level may be determined by processor 120 to be when the PLV standard error is below 0.03 arbitrary units of PLV. The PLV standard error may be calculated by processor 120 using a bootstrapping method. If the noise level is determined to not be low enough at step 420, more data is acquired at step 320. If the noise level is determined to be low enough, processor 120 proceeds to execute step 430.

If processor 120 determines that there is a significant response at step 340, then processor 120 also proceeds to execute step 430.

At step 430, processor 120 executes a decision rule. For example, step 430 may cause processor 120 to execute an adaptive decision procedure which produces the hearing estimate at step 440. In some embodiments, the adaptive procedure may be a staircase algorithm. In other embodiments, this procedure could be based on the Parametric Estimation by Sequential Testing (PEST) procedure (as described in Pollack, I. Perception & Psychophysics (1968) 3: 285. https://doi.org/10.3758/BF03212746).

The staircase algorithm is a rule-based method to determine the next stimulus level based upon the responses of one or more of the previous stimulus levels. As an example, a one-up one-down algorithm may be configured to cause processor 120 to increase the stimulus intensity at step 320 if, on the previous loop, no significant response is detected by processor 120 at step 340 and if the noise level is lower than a threshold at step 420. Conversely, the algorithm may cause processor 120 to decrease the stimulus intensity at step 320 if on the previous loop, a significant response is detected by step 340. After eight turning points, defined as when the stimulus intensity changed from increasing to decreasing or vice versa, the stimulus intensities at the last four of these turning points are averaged and the average is deemed the threshold estimate by processor 120 at step 440.

Further examples of staircase algorithms are described in Levitt (Levitt, H. C. C. H. (1971), Transformed up-down methods in psychoacoustics, The Journal of the Acoustical society of America, 49(2B), 467-477), the entirety of which is hereby incorporated by reference. Processor 120 executes a staircase algorithm to determine the next stimulus levels to test by interfacing with step 320. The staircase is initialised by processor 120 at the intensity determined at step 410. The step size X may be around 10 dB for embodiments for use with acoustic hearing devices, and around 1.5 dB for embodiments for use with electric hearing devices. The step size X may be decreased after the staircase has reached four turning points. According to some embodiments, the step size X may be decreased down to around 5 dB for embodiments for use with acoustic hearing devices, and to around 0.5 dB for embodiments for use with electric hearing devices.

FIG. 5 is a flowchart illustrating a method 500 for growth function threshold estimation using system 100. Method 500 may be executed by processor 120 and/or external processing device 195. According to some embodiments, method 500 may be performed by processor 120 executing automatic processing module 156.

Method 500 uses multiple features acquired at different stimulus intensities during methods 300 and 400 to extrapolate a hearing threshold for an individual.

Method 500 starts at step 510, where processor 120 initialises the stimulation level at the upper bound determined by processor 120 during method 300, or an upper bound that has been subjectively determined, for example from experience or from behavioural testing of stimulation intensity upper levels. Processor 120 causes a stimulus block to be sent at step 320, and receives a response at step 330, as described above with reference to method 300. The response may include at least one first response signal received post-stimulus, and at least one second response signal that does not correspond to a stimulus being delivered, or that corresponds to a stimulus at an intensity level that is below an expected hearing threshold for the subject, and therefore does not contain a response.

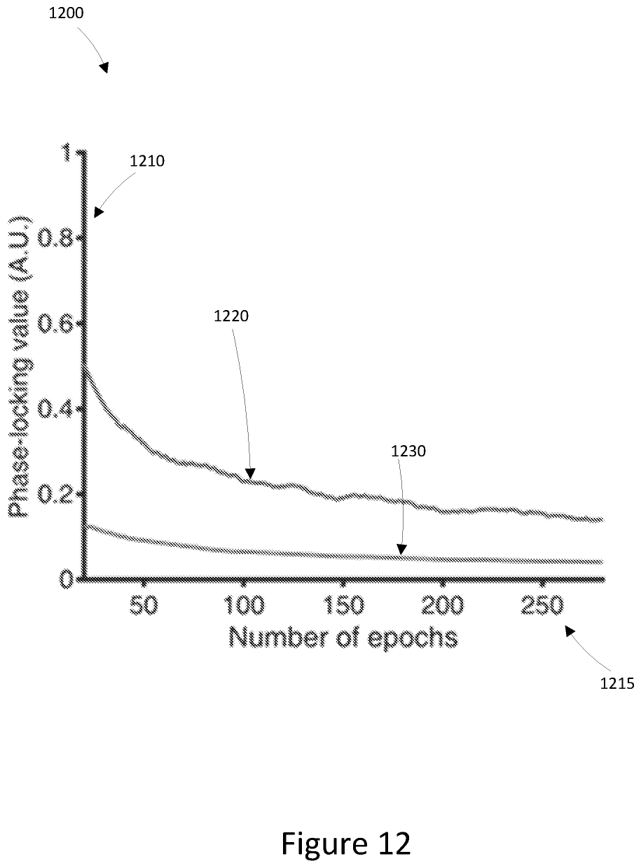

At step 520, at least one feature is extracted from the block of responses received from the current stimulus level, which may be the peak phase-locking value (PLV) feature. The window of extraction for the PLV feature may be 50 ms to 500 ms post-stimulus for the at least one first response signal in some embodiments, and may be from 1 to 20 Hz. In addition, at step 520, segments of the continuous recording that do not correspond to a stimulus being delivered or do not contain a response may be extracted from the recording by processor 120 as the at least one second response signal and used as a baseline response. For example, a segment from −600 ms to −150 ms pre-stimulus may be extracted and used as a baseline response. A baseline feature, which may be a baseline PLV feature, may be determined based on the baseline response. In some embodiments, the baseline responses are extracted so that the noise level is within 0.05 arbitrary units of PLV of the noise level of the response to the current stimulus. The noise level of the current stimulus-response may be calculated as the standard deviation of the bootstrapped feature distribution. In some embodiments, the noise level difference is within 0.01 arbitrary units of PLV. Ensuring that the noise levels for each response are of a similar value is necessary for a growth function to be correctly fitted. This is described in further detail below with reference to FIG. 12.

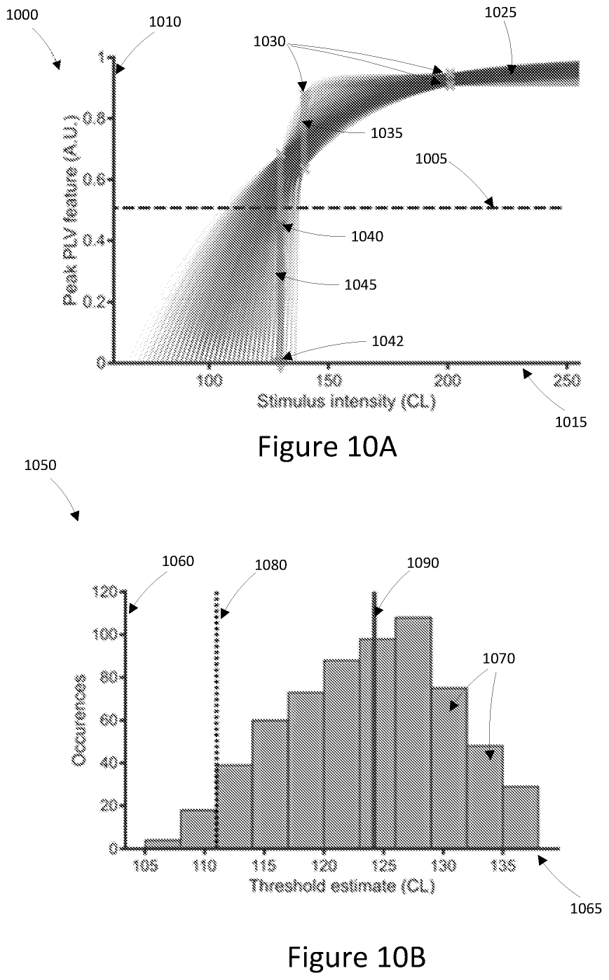

Processor 520 then executes step 530, at which processor 120 establishes upper and lower bounds of the features extracted at step 520. These bounds are proportionate to the uncertainty of each feature calculation at every stimulus intensity for which a response exists. In other words, the bounds are proportionate to the noise level of the current stimulus response. In some embodiments, the lower and upper bounds are one standard deviation away from the median of the bootstrap distribution of the features. In some embodiments, the lower and upper bounds are first and third quartiles of the bootstrap distribution of the features. In some embodiments, a lower bound that is below the baseline feature is replaced with negative infinity. This allows for features acquired at below-threshold intensities to not limit growth functions, which decreases exponentially with lower intensities. An example graph showing upper and lower bounds is described in further detail below with reference to FIG. 10A.

At step 540, processor 120 calculates a distribution of potential threshold estimates using the bounds calculated at step 530 and a defined parameter space, which may be described as the function

$$y = a ( 1 - {e}^{- \frac{x - b}{c}} ) .$$

In some embodiments, a may take on any value between 0 and 1 in steps of 0.01; b may take on any value between 0 and 200 in steps of 2; and c may take on any value between 2 and 100 in steps of 2. The parameter space may change depending on whether stimulation member 145 is configured to deliver acoustic or electric stimulation. For example, in an acoustic hearing application, b may take on any value between 0 to 100 in steps of 1. The parameter space may also change depending on the unit of conversion used by hearing assessment device 110 if using electric hearing, and the feature selected for processing. For example, a may take on any value between 0 and 30 in steps of 1 when using a peak-to-peak amplitude feature instead of phase-locking value feature.

The distribution of potential threshold estimates may be generated by processor 120 evaluating each growth function whose parameters a, b and c are described by the parameter space. In some embodiments, processor 120 may determine the x-axis value of the intersection between the baseline feature and each growth function of the parameter space to be one sample of the distribution of threshold estimates. In addition, processor 120 may eliminate or weight each growth function based on the bounds calculated at step 530. For example, in some embodiments, any growth function that exceeds the lower and upper bounds determined at step 530 may be excluded, as described below with reference to FIG. 10A. In some embodiments, each growth function's contribution is weighted by processor 120.

Some embodiments find the weighting U in two steps. First, an error term E is generated for each growth function in the parameter space. The squared difference between each value evaluated at stimulation intensity i G_{a,b,c}(i) and the corresponding real features F(i) determined by processor 120 from the data acquired during execution of method 500 is weighted and summed:

$${E}_{a , b , c} = {\sum }_{i} {( F ( i ) - {G}_{a , b , c} ( i ) )}^{2} * w ( i )$$

In one specific embodiments, the weighting function w may be calculated to be:

$$W = \{ \begin{matrix}0.008 , & p \geq 0.249 \\ - 16 {p}^{2} + 1 , & p < 0.249\end{matrix}$$

The p value may be the output of a statistical test, for example the Hotelling T² test, on whether the real features are significantly different from baseline.

Second, the growth functions of the parameter space are ranked by their error term E, and the one percent that have the lowest error terms are kept. The error values are then rescaled to a weight U by taking the best/lowest (E_{b}) and worst/highest (E_{w}) error terms and applying the following function below. The function scales the best growth function (smallest error) to a value of U=1, and the worst growth function (largest error after taking the best one percent) to a value of U=0.

$$U ( E ) = mE + c , \\ where m = \frac{1}{{E}_{b} - {E}_{w}} , \\ c = \frac{{E}_{w}}{{E}_{w} - {E}_{b}}$$

In some embodiments, each growth function's contribution is weighted by processor 120 by its feature value and its corresponding probability in the bootstrap distribution of real features from the acquired data. When all growth functions in the parameter space have been evaluated as described above, the threshold estimate from each is aggregated by processor 120 to a distribution, as described below with reference to FIG. 10B.

At step 550, processor 120 uses the threshold estimate distribution generated at step 540 to calculate a hearing threshold estimate and a measure of uncertainty of the estimate. In some embodiments, processor 120 determines the median of the distribution to be the threshold estimate, as described below with reference to FIG. 10B. In some embodiments, processor 120 determines the expected value of the distribution, which may be the average or mean value of the distribution, to be the threshold estimate. Processor 120 may determine the uncertainty of the estimate to be the standard deviation of the distribution.

At step 560, processor 120 determines whether the threshold estimate calculated at step 550 is the final threshold estimate based on whether a stopping criterion has been met. A stopping criterion may be when processor 120 determines that the uncertainty measure calculated in step 550 is below a set threshold, for example. In some embodiments, where electric hearing is used, the threshold may be 5 units in the hearing device's operating units. In some embodiments, where acoustic hearing is used, the threshold may be 10 dB. In some embodiments, a stopping criterion may be when a set number of trials or a set testing time is exceeded, when the otherwise measured quality of the signal exceeds a criterion, or may be subjectively determined. For example, according to some embodiments, the stopping criterion may be once 800 trials are performed, or after 20 minutes have elapsed.

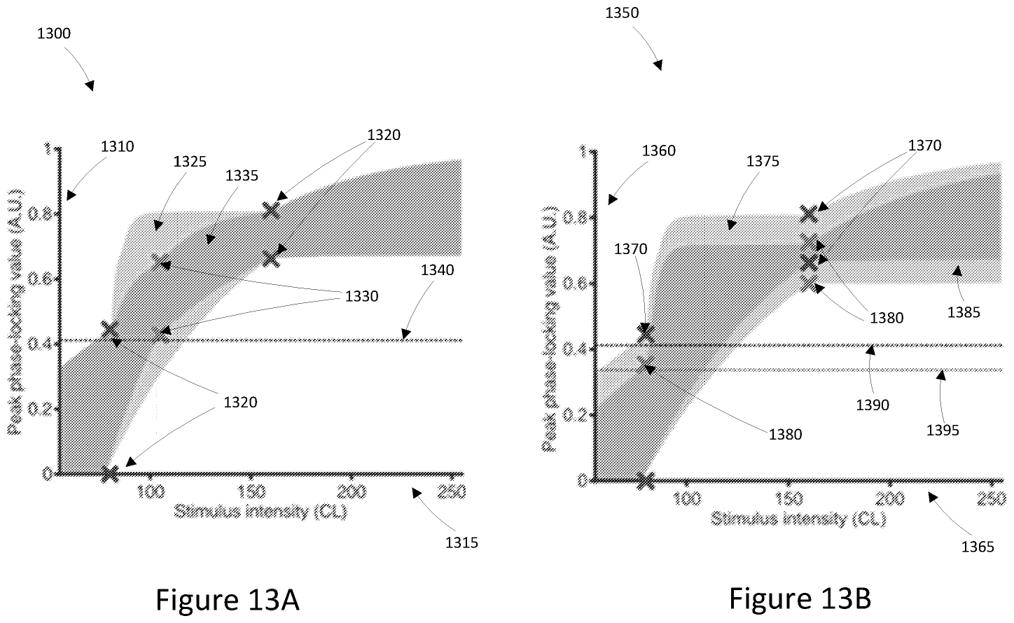

If the decision at step 560 is to continue testing, then processor 120 determines what data should be added at steps 570 and 580. The options may include adding epochs at a new stimulation intensity, or adding epochs to existing stimulation intensities. In some embodiments, processor 120 may determine that additional epochs should be added to ensure that the same number of epochs exist at every stimulation intensity, or that a similar number of epochs exist at every stimulation intensity. For example, according to some embodiments, processor 120 may determine that additional epochs should be added to ensure that the number of epochs at a given stimulation intensity is within 10 of the number of epochs at every other stimulation intensity. In some embodiments, processor 120 may determine that additional epochs should be added to ensure that the noise level of the captured data is within 0.05 arbitrary units of the PLV at every stimulation intensity. In some embodiments, processor 120 may determine that additional epochs should be added to ensure that the noise level difference is within 0.01 arbitrary units of PLV. To capture the optimal data, processor 120 performs step 570 to predict the response features or feature bounds for each possible next step, and then performs step 580 to use the predicted response features to choose an optimal next step. This is described in further detail below with reference to FIGS. 12, 13A and 13B.

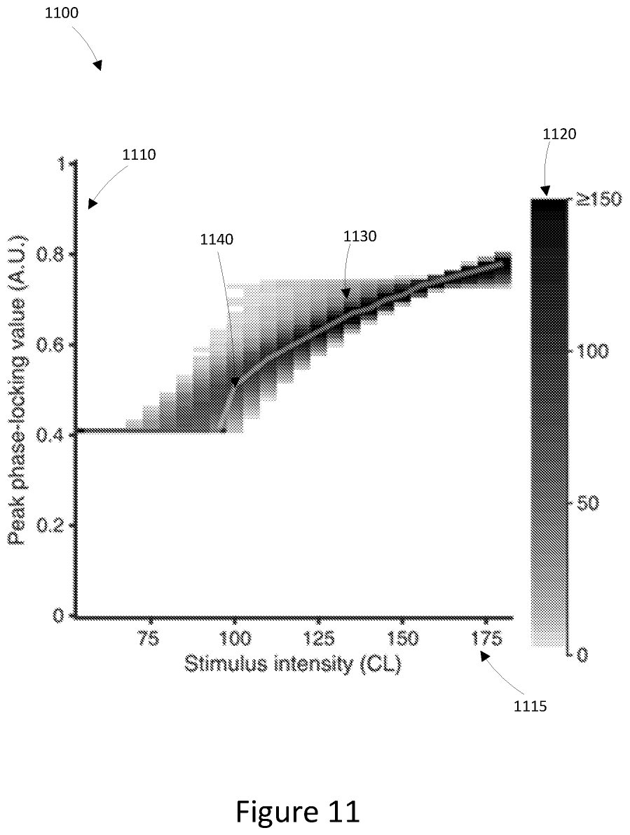

At step 570, processor 120 may perform two predictions which cover possible next steps. First, processor 120 predicts response features that would be captured by EEG module 170 if a new stimulation intensity is to be presented via stimulation member 145. The new stimulation intensity may be any level which has not already been presented, or a subset thereof. Second, processor 120 predicts new response features that would be captured by EEG module 170 if epochs are added to existing stimulation intensities. The prediction of response features at a new stimulation intensity may use the bounds that were generated at step 540. In some embodiments, the predicted response feature at a new intensity level may be determined by processor 120 to be the maximally-likely value of the distribution of growth functions, as described below with reference to FIG. 11. In some embodiments, the predicted response feature may be determined by processor 120 to be the median value of the distribution of growth functions. The prediction of response features when epochs are added to existing stimulation intensities may use a baseline distribution to scale the existing features and feature bounds.

An example baseline distribution is described below with reference to FIGS. 12 to 13B.

At step 580, processor 120 uses the predicted features and bounds from step 570 to evaluate the optimal next step. The growth function parameter space determined at step 540 may be used again, but with the predicted bounds calculated in step 570. Processor 120 may be configured to optimise the next step by minimising the number of growth functions that fit within the predicted bounds in some embodiments, as described below with reference to FIGS. 13A and 13B. In some embodiments, processor 120 may be configured to select an optimal step that minimises entropy or other uncertainty measures in the threshold estimate. In some embodiments, processor 120 may be configured to select an optimal step that minimises entropy or other uncertainty measures within the valid growth function parameter space. Once the optimal next step is determined by processor 120, processor 120 executes step 320 to acquire data at the selected stimulus intensity or intensities.

If processor 120 decides at step 560 to stop testing, then the threshold estimate 590 is determined to be the final threshold estimate found when step 550 was last executed.

In embodiments where the peak PLV feature is used, the parameter a is bounded between 0 and 1. The fitting may be performed by processor 120 using a least squares algorithm. Here, y indicates the feature values at each stimulus intensity, while x indicates the stimulus intensities, which in acoustic hearing is in dB SPL, and in electric hearing is in the hearing device's operating units. In some embodiments, more than one growth function may be fit by processor 120, and the function with the best goodness of fit, which may be quantified by processor 120 based on its adjusted r² value, is chosen by processor 120.

At step 530, processor 120 judges the chosen fit for validity. In some embodiments pertaining to a growth function of the form y=ax+b, the value of a must be positive and the adjusted r² value must also be positive. In some embodiments pertaining to a growth function of the form

$$y = a ( 1 - {e}^{- \frac{x - b}{c}} ) ,$$

the values of a and c must be positive and the adjusted r² value must also be positive. In some embodiments, the r² value must exceed a certain threshold, such as 0.7. If processor 120 determines that the fit is not valid at step 530, processor 120 proceeds to perform step 540 to acquire more data. If processor 120 determines that the fit is valid, processor 120 proceeds to perform step 550.

At step 540, processor 120 decides what additional data is required to improve the chosen growth function fit. Processor 120 may receive inputs including the already tested stimulus levels, the PLV feature values extracted in response to these stimulus levels, the difference between stimulus levels or their response features, the population average feature and the baseline features to make the determination at step 540. Processor 120 may execute a function that uses some or all of this input information to determine the number of additional trials required and the stimulus level at which to acquire the trials, in order to arrive at an improved growth function fit. The determined stimulus levels may be new stimulation levels, or processor 120 may determine that additional trials at an already-tested level should be performed. Once the parameters of any further trials are determined, processor 120 may move on to repeat steps 330 to 530, as described above.

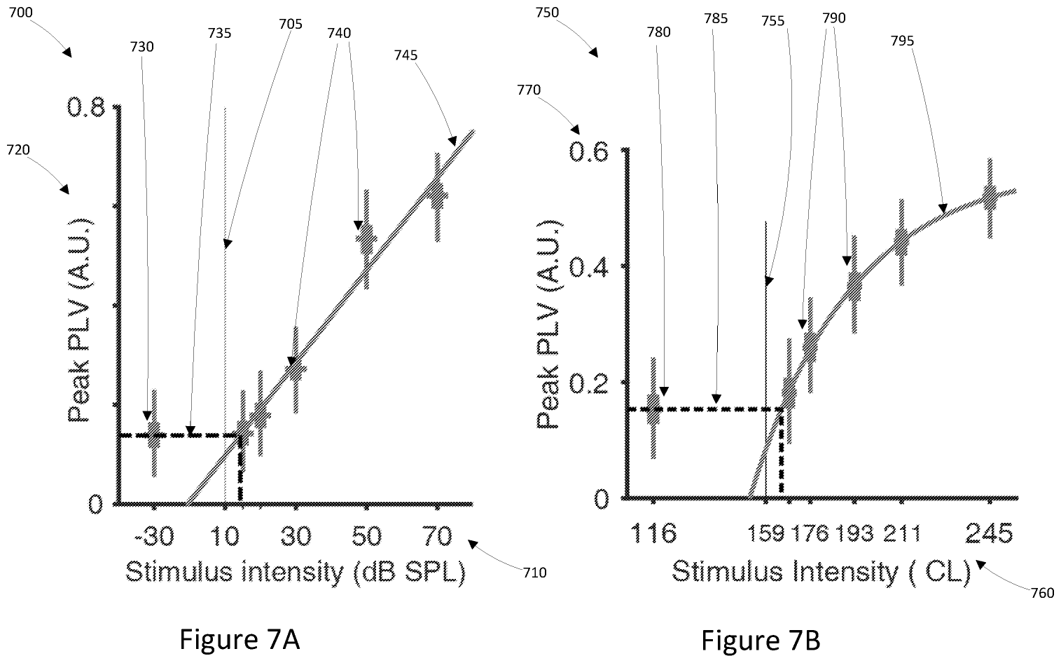

Once a growth fit function is found to be valid at step 530, processor 120 executes step 550 to extrapolate the threshold value. Processor 120 may execute an algorithm that uses the baseline feature determined at step 520 to extrapolate from the growth function. At step 560, processor 120 may use the corresponding stimulus intensity on an x-axis of a graph of the growth function to determine the threshold estimate, as described in further detail below with reference to FIGS. 7A and 7B.

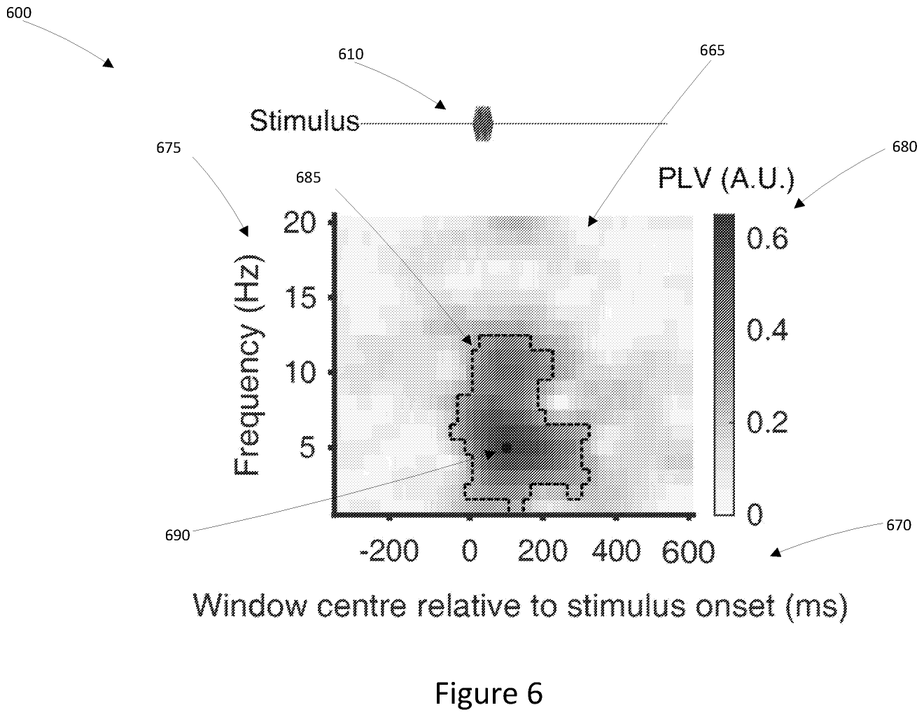

FIG. 6 shows an example frequency-domain spectrum representing the phase locking value (PLV) 665 measured in a subject when exposed to a 60 dB SL stimuli 600. The x-axis 670 shows the time to the stimulus onset, measured in milliseconds, while the y-axis 675 shows the frequency in Hz at which the PLV is calculated. The key 680 shows how the change in PLV is measured in a unit-less quantity and displayed as shading on graph 665. Black outline 685 encloses the region where the PLV is significantly higher than baseline. Significant in this example is determined to be when z>3 for a permutation test with baseline at −300 ms. Black dot 690 indicates the time-frequency location of the peak PLV, being the extracted feature values used for growth function fitting.

FIG. 7A shows an example graph 700 illustrating a growth function fitting procedure and threshold estimate using all epochs recorded from a single subject without a cochlear implant. Graph 700 has an x-axis 710 displaying a stimulation intensity in dB SPL, and a y-axis 720 displaying the peak PLV in an arbitrary unit. Data point 730 relates to a response to a sub-threshold stimulus.

Line 705 represents the subject's hearing threshold, 10.3 dB SPL, as determined by a behavioural test, used in this example case as a reference to compare with the identified threshold estimated by the methods and systems described herein. In using the described methods and systems to determine a hearing threshold, it is not necessary to determine line 705.

Data points 740 relate to significant responses to levels above the subject's hearing threshold. The horizontal bar, thick line and thin lines on data points 740 indicate the median, quartiles and the 1^{st}/99^{th} percentiles of the bootstrapped response data, respectively. In this example, the stimulation intensities are taken relative the subject's hearing threshold as determined by a behavioural test (line 705), but in using the described methods and systems to determine a hearing threshold, it is not necessary to determine line 705.

Extrapolated line 745 is the best fit linear growth function for data points 740. Extrapolated line 745 is calculated and extrapolated as described above for steps 550 and 560 of FIG. 5. The hearing threshold is the point on the x-axis which corresponds to the intersection between the regression line 745 and the baseline feature distribution 735, which is 14.6 dB SPL in the illustrated example, 4.3 dB higher than the hearing threshold as determined by the behavioural test.

FIG. 7B shows an example graph 750 illustrating a growth function fitting procedure and threshold estimate within a Nucleus device using all epochs recorded from a single subject with a cochlear implant. Graph 750 has an x-axis 760 displaying a stimulation intensity in CL, and a y-axis 770 displaying the peak PLV in an arbitrary unit. Data point 780 relates to a response to a sub-threshold stimulus.

Line 755 represents the subject's hearing threshold as determined by a behavioural test, used in this example case as a reference to compare with the identified threshold estimated by the methods and systems described herein. In using the described methods and systems to determine a hearing threshold, it is not necessary to determine line 755 by behavioural means.

Data points 790 relate to significant responses to levels above the subject's hearing threshold. The horizontal bar, thick line and thin lines on data points 790 indicate the median, quartiles and the 1^{st}/99^{th} percentiles of the bootstrapped response data, respectively.

Extrapolated line 795 is the best fit linear growth function for data points 790. Extrapolated line 795 is calculated and extrapolated as described above for steps 550 and 560 of FIG. 5. The hearing threshold is the point on the x-axis which corresponds to the intersection between the regression line 795 and the median of the baseline feature distribution 785, which is 165 CL in the illustrated example.

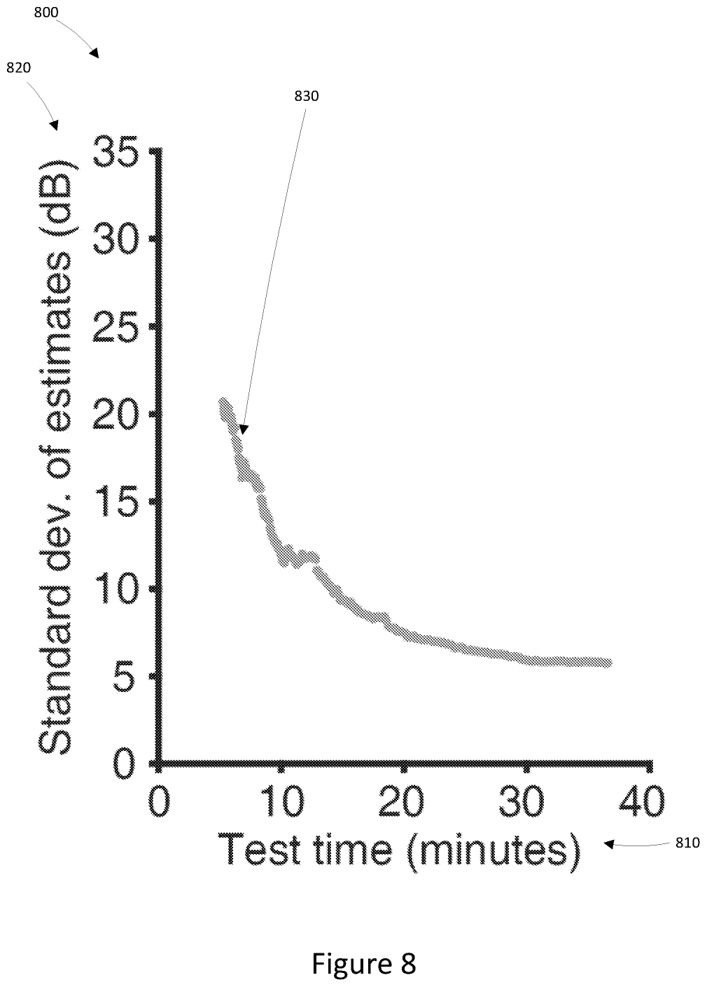

FIG. 8 shows a graph 800 illustrating the improvement in threshold estimation accuracy with an increase in test time when using the method described above with reference to FIGS. 3 to 5. X-axis 810 shows the test time in minutes, while y-axis 820 shows the standard deviation of estimates across test subjects in dB. As can be observed, the magnitude of the standard deviation of estimates 830 decreases as the test time increases, corresponding to an improvement in threshold accuracy.

While FIGS. 2 to 7B relate to using PLVs to determine a threshold estimate, according to some embodiments, another feature of a response signal may be used instead. For example, the peak-to-peak amplitude, RMS, or peak power could be used in place of the peak PLV to determine a hearing threshold.

FIG. 9A shows a graph 900 illustrating an example averaged time-domain EEG response 920 measured in a subject when exposed to 60 dB SL stimuli 905. The x-axis 910 shows the time to the stimulus onset, measured in milliseconds, while the y-axis 915 shows the measured response average in μV. Shaded error bars 925 enclose three standard errors of the mean of the response signal. In the illustrated embodiment, the peak to peak amplitude 935 and the root mean square (RMS) value 930 are extracted from a 50 ms to 500 ms post-stimulus window.

FIG. 9B shows a graph 950 illustrating an example frequency-domain spectrum representing the EEG response 985 measured in a subject when exposed to 60 dB SL stimuli 955. The x-axis 960 shows the time to the stimulus onset, measured in milliseconds, while the y-axis 965 shows the frequency of the response in Hz. The shading 970 on the response 985 represents the power change from a baseline power measurement, using an arbitrary unit (A.U.) of power. The value of the peak spectral power may be calculated between 1 to 20 Hz and 50 ms and 500 ms of the power spectrogram after normalising to the pre-stimulus baseline. Black outline 980 encloses the significant region of power change. Significant in this example is determined to be when z>3 for a permutation test with baseline at −300 ms. Black dot 975 indicates the time-frequency location of the peak power, being one of the extracted feature values used for growth function fitting.

The peak-to-peak amplitude, RMS and peak power as shown in FIGS. 9A and 9B may be used in threshold estimation as described above. For example, using method 400 as described above with reference to FIG. 4, the features can be used in conjunction with a suitable significance test to replace PLV as recited in step 340. In some embodiments, bootstrapping may be used for all features to test the significance of the features in the post-stimulus region compared to the pre-stimulus region. In some embodiments, spectral power can be tested for significance with the Wilcoxon rank-sum test between pre-stimulus spectra and post-stimulus spectra. Bootstrapping may also be used for all features to calculate the noise estimate in step 420 of method 400.

In another example, using method 500, as described above with reference to FIG. 5, features can be calculated after data acquisition and fit to a suitable growth function, providing an alternative to using PLV in step 520. In the case of peak-to-peak amplitude, root-mean-square and peak spectral power features, the exponential fitting parameter a of steps 530 and 550 need not be limited between 0 and 1 as for when using peak PLV.

FIG. 10A shows a graph 1000 illustrating an example of upper and lower feature bounds as determined by processor 120 at steps 530, 540 and 550 of method 500. Graph 1000 has an x-axis 1015 showing the stimulus intensity in current level (CL), and a y-axis 1010 showing the peak PLV feature in arbitrary units (AU). According to some embodiments, the stimulation intensity may be measured in current level for electric hearing applications, and sound pressure level (dB SPL) for acoustic hearing applications.

Line 1005 shows the level of the baseline feature determined by processor 120 at step 520 of method 500. Crosses 1030 and 1040 indicate upper and lower bounds calculated by processor 120 executing step 530 of method 500. Crosses 1040 located below line 1005 correspond to bounds falling below the baseline feature, which are extended to infinity 1042 by processor 120 at step 540 of method 500. Lines 1025 show all of the growth functions that fit within the bounds 1030. Any growth function that exceeds the lower and upper bounds 1030 and 1042 determined at step 530 are excluded.

FIG. 10B shows a graph 1050 showing how threshold estimates and uncertainty is derived by processor 120 at step 550 of method 500. Graph 1050 has an x-axis 1065 showing the threshold estimate in current level (CL), and a y-axis 1060 showing the number of occurrences of that threshold estimate based on the growth functions as shown in FIG. 10A. Histogram bars 1070 are determined by tallying the threshold estimate of each growth function determined by processor 120 at step 540 The threshold estimate distribution histogram may be determined as the CLs where each growth function 1025 crosses the baseline feature line 1005 in graph 1000 of FIG. 10A. Line 1090 is the threshold estimate as calculated from the histogram. The threshold estimate may be calculated as the median of the distribution of histogram bars 1070. Line 1080 is the behavioural hearing threshold, shown for reference.

FIG. 11 shows a graph 1100 illustrating how responses to untested stimulus intensities are predicted by processor 120 at step 570 of method 500. Graph 1100 has an x-axis 1115 showing the stimulus intensity in current level (CL), and a y-axis 1110 showing the peak PLV feature in arbitrary units (AU). Shading 1130 shows the weighted sum indicating the likelihood of response features at each stimulation intensity, with darker areas corresponding to a higher likelihood. Bar 1120 indicates the value of the weighted responses shown in shading 1130. Line 1140 indicates the maximum likelihood value for every stimulation intensity of graph 1100, and is taken as the predicted response.

FIG. 12: shows an example graph 1200 illustrating the change in the expected phase-locking feature baseline and noise level over a number of epochs as calculated by processor 120 during step 570 of method 500. Graph 1100 has an x-axis 1215 showing the number of epochs, and a y-axis 1210 showing the peak PLV feature in arbitrary units (AU). Line 1220 represents the expected phase-locking feature baseline changing as the number of epochs is increased, while line 1230 represents the noise level changing as the number of epochs is increased. The noise level may be determined based on the standard deviation of the bootstrap distribution. As lines 1220 and 1230 change with changing numbers of epochs, each peak PLV feature of the corresponding growth function must be generated within a predefined limit or an algorithm-limited range of acceptable epochs from each other peak PLV feature, to ensure that the growth function fit to the PLV features was not biased. For example, as described above, the number of epochs for each peak PLV feature may be within 10 epochs of each other peak PLV feature.

When epochs are added, processor 120 may scale the feature and feature bounds as previously determined during steps 530, 540 and 550 of method 500 by subtracting the amount the baseline is predicted to drop, as shown by line 1220, from the feature and feature bound values. Processor 120 may further scale the feature and feature bounds by multiplying the feature and feature bound values by the amount the bounds are predicted to shrink, as shown by line 1230. In addition, processor 120 may also scale the baseline feature as shown by line 1005 of FIG. 10A based on these rules.

FIG. 13A shows a graph 1300 showing an example of the predicted bounds determined by processor 120 for adding a new stimulus intensity at step 580 of method 500. Graph 1300 has an x-axis 1315 showing the stimulus intensity in current level (CL), and a y-axis 1310 showing the peak PLV feature in arbitrary units (AU).

Line 1340 indicates the baseline. Crosses 1320 indicate the original feature bounds, while crosses 1330 indicate the predicted feature bounds if a response is added at 105 CL. Shading 1325 shows the valid growth functions determined based on the original bounds 1320, while shading 1335 shows the valid growth functions once the predicted bounds 1330 are taken into account.

FIG. 13B shows a graph 1350 showing an example of the predicted bounds determined by processor 120 for adding epochs to existing stimulation intensities at step 580 of method 500. Graph 1350 has an x-axis 1365 showing the stimulus intensity in current level (CL), and a y-axis 1360 showing the peak PLV feature in arbitrary units (AU).

Line 1390 indicates the baseline, and line 1395 indicates the new baseline based on the predicted bounds added. Crosses 1370 indicate the original feature bounds, while crosses 1380 indicate the predicted feature bounds if a response is added based on 15 new epochs. Shading 1375 shows the valid growth functions determined based on the original bounds 1370, while shading 1385 shows the valid growth functions once the predicted bounds 1380 are taken into account.

In the case of processor 120 evaluating the results as shown in FIGS. 13A and 13B, the scenario as shown in FIG. 13B would be chosen as the next step, as the number of valid growth functions are minimised by adding additional epochs to existing stimulation intensities. At step 580 of method 500, processor 120 would therefore add additional epochs to the existing stimulation intensities.

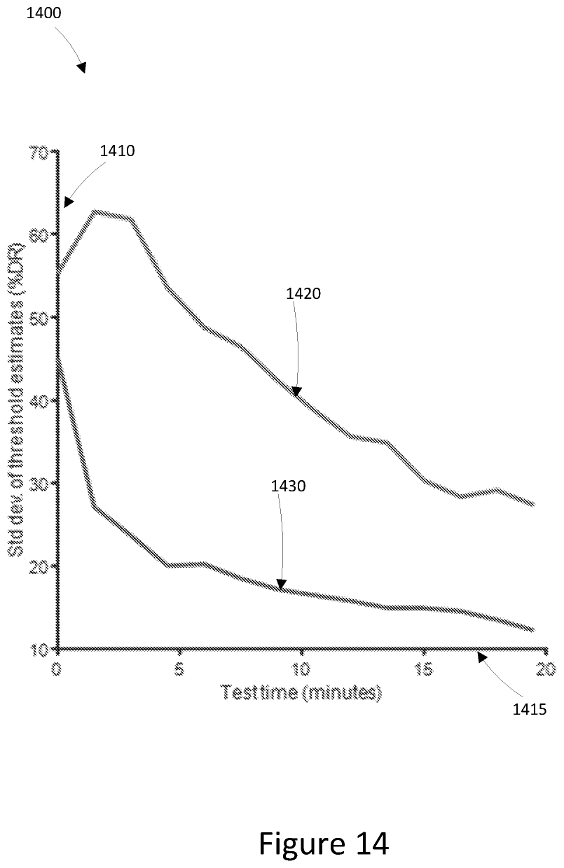

FIG. 14 shows an example graph 1400 illustrating the improvement in in threshold estimation accuracy with an increase in test time when using the method described above with reference to FIGS. 3 to 5. X-axis 1415 shows the test time in minutes, while y-axis 1410 shows the standard deviation of threshold estimates across test subjects in percent dynamic range (% DR), which is the unit of measure that would be used when stimulation device 135 is a cochlear implant. Line 1420 corresponds to results for a subject for whom no prior information exists, while line 1430 corresponds to results for a subject whose comfort level is known, as described above with reference to step 510 of method 500. As can be observed, the magnitude of the standard deviation of estimates decreases as the test time increases, corresponding to an improvement in threshold accuracy.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1-41. (canceled)

42. A method of determining a hearing threshold value for a subject, the method comprising:
receiving at least one first response signal relating to an aural stimulation experienced by the subject at a first intensity level;
receiving at least one second response signal relating to an aural stimulation experienced by the subject at a second intensity level, wherein the second intensity level is below an expected hearing threshold for the subject;
calculating at least one first value relating to the at least one first response signal;
calculating a second value relating to the at least one second response signal;
plotting the at least one first value against the first intensity level on a plot;
calculating a regression curve that fits the plot; and
determining the hearing threshold of the subject to be the intensity level that corresponds to the second value along the regression curve.

43. The method of claim 42, wherein the at least one first value is at least one of a phase-locking value (PLV), peak-to-peak amplitude, root mean square (RMS) value, or peak spectral power value of the at least one first response signal, and wherein the second value is at least one of a phase-locking value (PLV)), peak-to-peak amplitude, root mean square (RMS) value, or peak spectral power value of the at least one second response signal.

44. The method of claim 42, wherein the second intensity level corresponds to an absence of aural stimulation, and the second value is a baseline feature value.

45. The method of claim 44, wherein the baseline feature value is extracted so that the noise level of the baseline feature value is within 0.05 arbitrary units of PLV of the noise level of the aural stimulation.

46. The method of claim 44, further comprising establishing upper and lower bounds of the at least one first value, wherein the upper and lower bounds are proportionate to the noise level of the at least one first response.

47. The method of claim 46, further comprising modifying the upper and lower bounds based on their proximity to the baseline feature value.

48. The method of claim 46, further comprising replacing any lower bounds that are below the baseline feature value with negative infinity.

49. The method of claim 46, wherein the step of calculating a regression curve that fits the plot comprises calculating at least one growth function within a defined parameter space.

50. The method of claim 46, further comprising applying a weight to each growth function based on the upper and lower bounds.

51. The method of claim 42, wherein the at least one first response signal and the at least one second response signal relate to electrical activity of the subject's brain.

52. The method of claim 42, further comprising delivering the aural stimulation.

53. The method of claim 52, wherein the aural stimulation is delivered for a period of between 5 ms and 100 ms.

54. The method of claim 52, wherein the aural stimulation is delivered at a first intensity level of between −10 dB SPL and 120 dB SPL.

55. The method of claim 42, wherein the second intensity level is delivered for a period of between 0.1 seconds and 10 seconds.

56. The method of claim 42, wherein receiving at least one first response signal relating to an aural stimulation experienced by the subject at a first intensity level comprises receiving a plurality of first response signals relating to aural stimulation experienced by the subject at a plurality of different intensity levels.

57. The method of claim 42, further comprising repeating the steps of the method with additional epochs of response signals until a stopping criterion is met.

58. The method of claim 57, wherein the additional epochs are added to ensure that the noise level is within 0.05 arbitrary units of PLV at every stimulation intensity

59. A system for determining a hearing threshold value for a subject, the system comprising:
a hearing assessment device configured to:
receive at least one first response signal relating to an aural stimulation experienced by the subject at a first intensity level;
receive at least one second response signal relating to an aural stimulation experienced by the subject at a second intensity level, wherein the second intensity level is below an expected hearing threshold for the subject;
calculate at least one first value relating to the at least one first response signal;
calculate a second value relating to the at least one second response signal;
plot the at least one first value against the first intensity level on a plot;
calculate a regression curve that fits the plot; and
determine the hearing threshold of the subject to be the intensity level that corresponds to the second value along the regression curve.

60. The system of claim 59, further comprising a stimulation member configured to provide the aural stimulation.

61. The system of claim 59, further comprising at least two electrodes configured to measure the at least one first response signal and the at least second first response signal and communicate the at least one first response signal and the at least second first response signal to the hearing assessment device.
